SEQUENCE LISTING (3 sequences)

SEQ ID NO: 1 - protein, 155 aa, Artificial Sequence
  Artificial Pti5-like sequence
MFSQSQSDLPLNENDSQDMVIYQVLNEANALHNTYLPQRNQLLPNGLEPTRNIAKKYRGVRRRPWGKYAAEIRDSARHGARVWLGTFETAEEAALAYDRAAFKMRGAKALLNFPAEIVAASSSSLSSNSYTNNNNSNSSSSESSSGSSCTFNSRI

SEQ ID NO: 2 - protein, 86 aa, Artificial Sequence
  Artificial CaSAR8.2A-like sequence
MVSKNNIFICLSLIILLIMSSQIVAREMNSEAPAPLTQAMNGNNISEAKGVGRHLVKGLGKIFRAGKVIYCKTCKTCYGLCDYCCV

SEQ ID NO: 3 - protein, 639 aa, Artificial Sequence
  Artificial RLK2-like sequence
MRLYVELLLLSLVLGFFVSCDAFPSNEVEALNTFKEAIYEDPLLVLSNWNALDSDPCDWAGITCSMSRDHVIKINISGASLKGFLAPELGQITYLQELILHGNILIGTIPKEIGLLKNLKVLDLGMNQLTGPIPPEIGNLTNIMKINLQSNGLTGKLPPELGNLKYLQELRLDRNKLQGSVPFTSNTHGMYASGANPTGFCRSSQLKVADFSYNFLVGSIPKCLGYLPRSSFQGNCLQTKDPKQRPSSQCAKSHAATSKKKHSASKPTWLLALEIVTGVMVGSLFLVALLTALQRCNNKSSIIIPWKKSGSEKDHMTVYVDSEMLKDVVRYSRQELEVACEDFSNIIGSSPDSLVYKGTMKGGPEIAVISLCVKEEHWTGYLELYFQREVADLARLNHENTGKLLGYCRESTPFTRMLVFEYASNGTLYEHLHYGEGCQLSWTRRMKIVIGIARGLKYLHTELDPPFTISELNSNAVYLTEDFSPKLVDFESWKTILSRSEKNSGAISSEGAICVLPNSLEHRHLDIQGNIYAFGVLLLEIISGRPPYCKDKGCLVDWAKEYLEAPEVMSYVVDPELKHFRQEDLKVICEVISLCIHPDPSDRPSMKELCAMLESRIDTSISAELKASSLAWAELALSS

(19) United States
(12) Patent Application Publication
(10) Pub. No.: US 2024/0026372 A1
(43) Pub. Date: Jan. 25, 2024

(54) PLANT YIELD IMPROVEMENT

(71) Applicant: BASF SE, Ludwigshafen (DE)

(72) Inventors: Yunxing Cory Cui, Research Triangle Park, NC (US); Brody John De Young, Research Triangle Park, NC (US); Holger Schultheiss, Limburgerhof (DE)

(21) Appl. No.: 18/023,397

(22) PCT Filed: Aug. 30, 2021
(86) PCT No.: PCT/EP2021/073904

§ 371 Date: Feb. 27, 2023

Related U.S. Application Data
(60) Provisional application No. 63/072,742, filed on Aug. 31, 2020.

(51) Int. Cl. C12N 15/82

(52) U.S. Cl. CPC C12N 15/8282

(57) ABSTRACT

The present invention relates to plant breeding and farming. In particular the invention relates to materials and methods for improving plant yield, especially of soybean. Preferably such improvement is visible under fungal pathogen stress, caused e.g. by soybean rust. Methods of the invention comprise cultivating a plant comprising a heterologous expression cassette comprising a gene selected from Pti5, SAR8.2 and RLK2.

### FIELD OF THE INVENTION

The present invention relates to plant breeding and farming. In particular the invention relates to materials and methods for improving plant yield. Preferably such improvement is visible under fungal pathogen stress.

### BACKGROUND OF THE INVENTION

Plant pathogenic organisms and particularly fungi have resulted in severe reductions in crop yield in the past, in worst cases leading to famine. Monocultures in particular are highly susceptible to an epidemic-like spreading of diseases. To date, the pathogenic organisms have been controlled mainly by using pesticides. Currently the possibility of directly modifying the genetic disposition of a plant or pathogen is also open to man. Alternatively, naturally occurring fungicides produced by the plants after fungal infection can be synthesized and applied to the plants.

The term “resistance” as used herein refers to an absence or reduction of one or more disease symptoms in a plant caused by a plant pathogen. Resistance generally describes the ability of a plant to prevent, or at least curtail the infestation and colonization by a harmful pathogen. Different mechanisms can be discerned in the naturally occurring resistance, with which the plants fend off colonization by phytopathogenic organisms (Schopfer and Brennicke (1999) Pflanzenphysiologie, Springer Verlag, Berlin-Heidelberg, Germany).

With regard to resistance, a differentiation is made between compatible and incompatible interactions. In the compatible interaction, an interaction occurs between a virulent pathogen and a susceptible plant. The pathogen survives and may build up reproductive structures, while the host is seriously hampered in development or dies off. An incompatible interaction occurs, on the other hand, when the pathogen infects the plant but is inhibited in its growth before or after weak development of symptoms (mostly by the presence of Resistance (R) genes of the NBS-LRR family, see below). In the latter case, the plant is resistant to the respective pathogen (Schopfer and Brennicke, vide supra). However, this type of resistance is mostly specific for a certain strain or pathogen.

In both compatible and incompatible interactions, a defensive and specific reaction of the host to the pathogen occurs. In nature, however, this resistance is often overcome because of the rapid evolutionary development of new virulent races of the pathogens (Neu et al. (2003) American Cytopathol. Society, MPMI 16 No. 7: 626-633).

Most pathogens are plant species specific. This means that a pathogen can induce a disease in a certain plant species, but not in other plant species (Heath (2002) Can. J. Plant Pathol. 24: 259-264). The resistance against a pathogen in certain plant species is called non-host resistance. The non-host resistance offers strong, broad, and permanent protection from phytopathogens. Genes providing non-host resistance provide the opportunity of a strong, broad and permanent protection against certain diseases in non-host plants. In particular, such a resistance works for different strains of the pathogen.

Fungi are distributed worldwide. Approximately 100 000 different fungal species are known to date. Thereof, rusts are of great importance. They can have a complicated development cycle with up to five different spore stages (spermatium, aecidiospore, uredospore, teleutospore and basidiospore).

During the infection of plants by pathogenic fungi, different phases are usually observed. The first phases of the interaction between phytopathogenic fungi and their potential host plants are decisive for the colonization of the plant by the fungus. During the first stage of the infection, the spores become attached to the surface of the plants, germinate, and the fungus penetrates the plant. Fungi may penetrate the plant via existing ports such as stomata, lenticels, hydathodes and wounds, or else they penetrate the plant epidermis directly as the result of mechanical force with the aid of cell wall digesting enzymes. Specific infection structures are developed for penetration of the plant. To counteract, plants have developed physical barriers, such as wax layers, and chemical compounds having antifungal effects to inhibit spore germination, hyphal growth or penetration.

The soybean rust Phakopsora pachyrhizi directly penetrates the plant epidermis. After growing through the epidermal cell, the fungus reaches the intercellular space of the mesophyll, where the fungus starts to spread through the leaf. To acquire nutrients, the fungus penetrates mesophyll cells and develops haustoria inside the mesophyll cells. During the penetration process the plasma membrane of the penetrated mesophyll cell stays intact.

It is a particularly troubling feature of Phakopsora rusts that these pathogens exhibit an immense variability, thereby overcoming novel plant resistance mechanisms and novel fungicide activities within a few years and sometimes already within one Brazilian growing season.

Fusarium species are important plant pathogens that attacks a wide range of plant species including many important crops such as maize and wheat. They cause seed rots and seedling blights as well as root rots, stalk rots and ear rots. Pathogens of the genus Fusarium infect the plants via roots, silks or previously infected seeds or they penetrate the plant via wounds or natural openings and cracks. After a very short establishment phase the Fusarium fungi start to secrete mycotoxins such as trichothecenes, zearalenone and fusaric acid into the infected host tissues leading to cell death and maceration of the infected tissue. Feeding on dead tissue, the fungus then starts to spread through the infected plant leading to severe yield losses and decreases in quality of the harvested grain.

Biotrophic phytopathogenic fungi depend for their nutrition on the metabolism of living plant cells. This type of fungi belongs to the group of biotrophic fungi, like many rust fungi, powdery mildew fungi or oomycete pathogens like the genus Phytophthora or Peronospora. Necrotrophic phytopathogenic fungi depend for their nutrition on dead cells of the plants, e.g. species from the genus Fusarium, Rhizoctonia or Mycospaerella. Soybean rust occupies an intermediate position. It it penetrates the epidermis directly, whereupon the penetrated cell becomes necrotic. However, after penetration, the fungus changes over to an obligate-biotrophic lifestyle. The subgroup of the biotrophic fungal pathogens which follows essentially such an infection strategy are heminecrotrophic.

Yield is affected by various factors, for example the number and size of the plant organs, plant architecture (for example, the number of branches), number of filled seed or grains, plant vigor, growth rate, root development, utilization of water and nutrients and stress tolerance. In the past efforts have been made to create plants resistant against fungal pathogens.

With some surprise and disappointment it has been found that improvements in fungal resistance are not correlated to improvements in yield. In particular, even genes which reliably lead to a strong fungal resistance may not increase, or may even decrease, yield.

Contrary to common sense and speculations and assertions in literature, the traits of fungal resistance and yield are in the best case independent from each other, but often even counteracting (for a review on this topic see Ning et al. Balancing Immunity and Yield in Crop Plants Trends in Plant Science 22(12), 1069-1079). Farmers, however, are mainly interested in yield, the degree of plants unaffected by fungal infections is of no concern unless yield is affected.

It was thus the object of the invention to provide materials and methods to improve plant yield, particularly for crops and preferably providing yield increases despite potential fungal pathogen stress. In particular it was a preferred object of the invention to provide materials and methods which lead to plant material of heritably improved yield even under conditions of infection by a fungal pathogen, preferably a rust fungus and most preferably a rust fungus of genus Phakopsora, but also under conditions without significant infection pressure.

### SUMMARY OF THE INVENTION

It has now been found that certain genes provide yield improvements in plants, in particular in crops. Thus, the following teachings of the invention are encompassed by the disclosure:

The invention provides a method for improving the yield produced by a plant relative to a control plant, comprising
i) providing a plant comprising a heterologous expression cassette comprising a gene selected from Pti5, SAR8.2 and RLK2, and
ii) cultivating the plant.

Correspondingly the invention provides the use of a gene selected from Pti5, SAR8.2 and RLK2 for improving yield of a plant.

The invention also provides a farming method for improving the yield produced by a plant relative to a control plant, comprising cultivation of a plant comprising a heterologous expression cassette comprising a gene selected from Pti5, SAR8.2 and RLK2, wherein during cultivation of the plant the number of pesticide treatments per growth season is reduced by at least one relative to the control plant, preferably by at least two.

Furthermore the invention provides a method for producing a hybrid plant having improved yield relative to a control plant, comprising
i) providing a first plant material comprising a heterologous expression cassette comprising a gene selected from Pti5, SAR8.2 and RLK2, and a second plant material not comprising said heterologous expression cassette,
ii) producing an F1 generation from a cross of the first and second plant material, and
iii) selecting one or more members of the F1 generation that comprises said heterologous expression cassette.

### BRIEF DESCRIPTION OF FIGURES

FIG. 1 shows a comparison of soybean yield (determined according to example 4) versus relative fungal resistance (determined according to example 3).

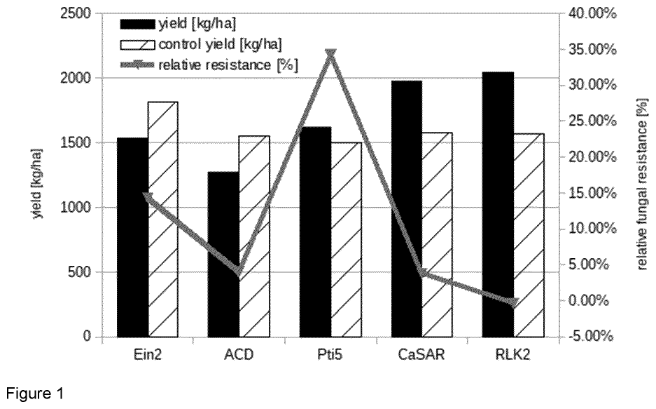

### BRIEF DESCRIPTION OF SEQUENCES

| | |
|---|---|
| SEQ ID. | nt/aa description |

| | | |
|---|---|---|
| 1 | aa | artificial Pti5-like sequence |
| 2 | aa | artificial CaSAR8.2A-like sequence |
| 3 | aa | artificial RLK2-like sequence |

### DETAILED DESCRIPTION OF THE INVENTION

The technical teaching of the invention is expressed herein using the means of language, in particular by use of scientific and technical terms. However, the skilled person understands that the means of language, detailed and precise as they may be, can only approximate the full content of the technical teaching, if only because there are multiple ways of expressing a teaching, each necessarily failing to completely express all conceptual connections, as each expression necessarily must come to an end. With this in mind the skilled person understands that the subject matter of the invention is the sum of the individual technical concepts signified herein or expressed, necessarily in a pars-pro-toto way, by the innate constrains of a written description. In particular, the skilled person will understand that the signification of individual technical concepts is done herein as an abbreviation of spelling out each possible combination of concepts as far as technically sensible, such that for example the disclosure of three concepts or embodiments A, B and C are a shorthand notation of the concepts A+B, A+C, B+C, A+B+C. In particular, fallback positions for features are described herein in terms of lists of converging alternatives or instantiations. Unless stated otherwise, the invention described herein comprises any combination of such alternatives. The choice of more or less preferred elements from such lists is part of the invention and is due to the skilled person's preference for a minimum degree of realization of the advantage or advantages conveyed by the respective features. Such multiple combined instantiations represent the adequately preferred form(s) of the invention.

In so far as recourse herein is made to entries in public databases, for example Uniprot and PFAM, the contents of these entries are those as of 2020 May 20. Unless stated to the contrary, where the entry comprises a nucleic acid or amino acid sequence information, such sequence information is incorporated herein.

As used herein, terms in the singular and the singular forms like “a”, “an” and “the” include plural referents unless the content clearly dictates otherwise. Thus, for example, use of the term “a nucleic acid” optionally includes, as a practical matter, many copies of that nucleic acid molecule; similarly, the term “probe” optionally (and typically) encompasses many similar or identical probe molecules. Also as used herein, the word “comprising” or variations such as “comprises” or “comprising” will be understood to imply the inclusion of a stated element, integer or step, or group of elements, integers or steps, but not the exclusion of any other element, integer or step, or group of elements, integers or steps.

As used herein, the term “and/or” refers to and encompasses any and all possible combinations of one or more of the associated listed items, as well as the lack of combinations when interpreted in the alternative (“or”). The term “comprising” also encompasses the term “consisting of”.

The term “about”, when used in reference to a measurable value, for example an amount of mass, dose, time, temperature, sequence identity and the like, refers to a variation of 0.1%, 0.25%, 0.5%, 0.75%, 1%, 2%, 3%, 4%, 5%, 6%, 7%, 8%, 9%, 10%, 15% or even 20% of the specified value as well as the specified value. Thus, if a given composition is described as comprising “about 50% X,” it is to be understood that, in some embodiments, the composition comprises 50% X whilst in other embodiments it may comprise anywhere from 40% to 60% X (i.e., 50%±10%).

As used herein, the term “gene” refers to a biochemical information which, when materialised in a nucleic acid, can be transcribed into a gene product, i.e. a further nucleic acid, preferably an RNA, and preferably also can be translated into a peptide or polypeptide. The term is thus also used to indicate the section of a nucleic acid resembling said information and to the sequence of such nucleic acid (herein also termed “gene sequence”).

Also as used herein, the term “allele” refers to a variation of a gene characterized by one or more specific differences in the gene sequence compared to the wild type gene sequence, regardless of the presence of other sequence differences. Alleles or nucleotide sequence variants of the invention have at least, in increasing order of preference, 30%, 40%, 50%, 60%, 70%, 71%, 72%, 73%, 74%, 75%, 76%, 77%, 78%, 79%, 80%, 81%-84%, 85%, 86%, 87%, 88%, 89%, 90%, 91%, 92%, 93%, 94%, 95%, 96%, 97%, 98% or 99% nucleotide “sequence identity” to the nucleotide sequence of the wild type gene. Correspondingly, where an “allele” refers to the biochemical information for expressing a peptide or polypeptide, the respective nucleic acid sequence of the allele has at least, in increasing order of preference, 30%, 40%, 50%, 60%, 70%, 71%, 72%, 73%, 74%, 75%, 76%, 77%, 78%, 79%, 80%, 81%-84%, 85%, 86%, 87%, 88%, 89%, 90%, 91%, 92%, 93%, 94%, 95%, 96%, 97%, 98% or 99% amino acid “sequence identity” to the respective wild type peptide or polypeptide.

Protein or nucleic acid variants may be defined by their sequence identity when compared to a parent protein or nucleic acid. Sequence identity usually is provided as “% sequence identity” or “% identity”. To determine the percent-identity between two amino acid sequences in a first step a pairwise sequence alignment is generated between those two sequences, wherein the two sequences are aligned over their complete length (i.e., a pairwise global alignment). The alignment is generated with a program implementing the Needleman and Wunsch algorithm (J. Mol. Biol. (1979) 48, p. 443-453), preferably by using the program “NEEDLE” (The European Molecular Biology Open Software Suite (EMBOSS)) with the programs default parameters (gapopen=10.0, gapextend=0.5 and matrix=EBLOSUM62). The preferred alignment for the purpose of this invention is that alignment, from which the highest sequence identity can be determined.

The following example is meant to illustrate two nucleotide sequences, but the same calculations apply to protein sequences:
Seq A: AAGATACTG length: 9 bases
Seq B: GATCTGA length: 7 bases

Hence, the shorter sequence is sequence B.

Producing a pairwise global alignment which is showing both sequences over their complete lengths results in

Seq A: AAGATACTG-
||| |||
Seq B: --GAT-CTGA

The “|” symbol in the alignment indicates identical residues (which means bases for DNA or amino acids for proteins). The number of identical residues is 6.

The “-” symbol in the alignment indicates gaps. The number of gaps introduced by alignment within the sequence B is 1. The number of gaps introduced by alignment at borders of sequence B is 2, and at borders of sequence A is 1.

The alignment length showing the aligned sequences over their complete length is 10.

Producing a pairwise alignment which is showing the shorter sequence over its complete length according to the invention consequently results in:

Seq A: GATACTG-
||| |||
Seq B: GAT-CTGA

Producing a pairwise alignment which is showing sequence A over its complete length according to the invention consequently results in:

Seq A: AAGATACTG
||| |||
Seq B: --GAT-CTG

Producing a pairwise alignment which is showing sequence B over its complete length according to the invention consequently results in:

Seq A: GATACTG-
||| |||
Seq B: GAT-CTGA

The alignment length showing the shorter sequence over its complete length is 8 (one gap is present which is factored in the alignment length of the shorter sequence).

Accordingly, the alignment length showing sequence A over its complete length would be 9 (meaning sequence A is the sequence of the invention), the alignment length showing sequence B over its complete length would be 8 (meaning sequence B is the sequence of the invention).

After aligning the two sequences, in a second step, an identity value shall be determined from the alignment. Therefore, according to the present description the following calculation of percent-identity applies: %-identity=(identical residues/length of the alignment region which is showing the respective sequence of this invention over its complete length)*100. Thus, sequence identity in relation to comparison of two amino acid sequences according to the invention is calculated by dividing the number of identical residues by the length of the alignment region which is showing the respective sequence of this invention over its complete length. This value is multiplied with 100 to give “%-identity”. According to the example provided above, %-identity is: for sequence A being the sequence of the invention (6/9)*100=66.7%; for sequence B being the sequence of the invention (6/8)*100=75%.

The term “hybridisation” as defined herein is a process wherein substantially complementary nucleotide sequences anneal to each other. The hybridisation process can occur entirely in solution, i.e. both complementary nucleic acids are in solution. The hybridisation process can also occur with one of the complementary nucleic acids immobilised to a matrix such as magnetic beads, Sepharose beads or any other resin. The hybridisation process can furthermore occur with one of the complementary nucleic acids immobilised to a solid support such as a nitrocellulose or nylon membrane or immobilised by e.g. photolithography to, for example, a siliceous glass support (the latter known as nucleic acid arrays or microarrays or as nucleic acid chips). In order to allow hybridisation to occur, the nucleic acid molecules are generally thermally or chemically denatured to melt a double strand into two single strands and/or to remove hairpins or other secondary structures from single stranded nucleic acids.

The term “stringency” refers to the conditions under which a hybridisation takes place. The stringency of hybridisation is influenced by conditions such as temperature, salt concentration, ionic strength and hybridisation buffer composition. Generally, low stringency conditions are selected to be about 30° C. lower than the thermal melting point (Tm) for the specific sequence at a defined ionic strength and pH. Medium stringency conditions are when the temperature is 20° C. below Tm, and high stringency conditions are when the temperature is 10° C. below Tm. High stringency hybridisation conditions are typically used for isolating hybridising sequences that have high sequence similarity to the target nucleic acid sequence. However, nucleic acids may deviate in sequence and still encode a substantially identical polypeptide, due to the degeneracy of the genetic code. Therefore, medium stringency hybridisation conditions may sometimes be needed to identify such nucleic acid molecules.

The “Tm” is the temperature under defined ionic strength and pH, at which 50% of the target sequence hybridises to a perfectly matched probe. The Tm is dependent upon the solution conditions and the base composition and length of the probe. For example, longer sequences hybridise specifically at higher temperatures. The maximum rate of hybridisation is obtained from about 16° C. up to 32° C. below Tm. The presence of monovalent cations in the hybridisation solution reduce the electrostatic repulsion between the two nucleic acid strands thereby promoting hybrid formation; this effect is visible for sodium concentrations of up to 0.4M (for higher concentrations, this effect may be ignored). Formamide reduces the melting temperature of DNA-DNA and DNA-RNA duplexes with 0.6 to 0.7° C. for each percent formamide, and addition of 50% formamide allows hybridisation to be performed at 30 to 45° C., though the rate of hybridisation will be lowered. Base pair mismatches reduce the hybridisation rate and the thermal stability of the duplexes. On average and for large probes, the Tm decreases about 1° C. per % base mismatch. The Tm may be calculated using the following equations, depending on the types of hybrids:
DNA-DNA hybrids (Meinkoth and Wahl, Anal. Biochem., 138: 267-284, 1984): Tm=81.5° C.+16.6×log([Na+]{a})+0.41×%[G/C{b}]−500×[L{c}]−1−0.61×% formamide
DNA-RNA or RNA-RNA hybrids: Tm=79.8+18.5 (log 10[Na+]{a})+0.58 (% G/C{b})+11.8 (% G/C{b})2−820/L{c}
oligo-DNA or oligo-RNAd hybrids:
for <20 nucleotides: Tm=2 ({ln})
for 20-35 nucleotides: Tm=22+1.46 ({ln})

wherein:
{a} or for other monovalent cation, but only accurate in the 0.01-0.4 M range
{b} only accurate for % GC in the 30% to 75% range
{c} L=length of duplex in base pairs
{d} Oligo, oligonucleotide
{ln} effective length of primer=2×(no. of G/C)+(no. of A/T)

Non-specific binding may be controlled using any one of a number of known techniques such as, for example, blocking the membrane with protein containing solutions, additions of heterologous RNA, DNA, and SDS to the hybridisation buffer, and treatment with Rnase. For non-related probes, a series of hybridizations may be performed by varying one of (i) progressively lowering the annealing temperature (for example from 68° C. to 42° C.) or (ii) progressively lowering the formamide concentration (for example from 50% to 0%). The skilled artisan is aware of various parameters which may be altered during hybridisation and which will either maintain or change the stringency conditions.

Besides the hybridisation conditions, specificity of hybridisation typically also depends on the function of post-hybridisation washes. To remove background resulting from non-specific hybridisation, samples are washed with dilute salt solutions. Critical factors of such washes include the ionic strength and temperature of the final wash solution: the lower the salt concentration and the higher the wash temperature, the higher the stringency of the wash. Wash conditions are typically performed at or below hybridisation stringency. A positive hybridisation gives a signal that is at least twice of that of the background. Generally, suitable stringent conditions for nucleic acid hybridisation assays or gene amplification detection procedures are as set forth above. More or less stringent conditions may also be selected. The skilled artisan is aware of various parameters which may be altered during washing and which will either maintain or change the stringency conditions.

For example, typical high stringency hybridisation conditions for DNA hybrids longer than 50 nucleotides encompass hybridisation at 65° C. in 1×SSC or at 42° C. in 1×SSC and 50% formamide, followed by washing at 65° C. in 0.3×SSC. Examples of medium stringency hybridisation conditions for DNA hybrids longer than 50 nucleotides encompass hybridisation at 50° C. in 4×SSC or at 40° C. in 6×SSC and 50% formamide, followed by washing at 50° C. in 2×SSC. The length of the hybrid is the anticipated length for the hybridising nucleic acid. When nucleic acids of known sequence are hybridised, the hybrid length may be determined by aligning the sequences and identifying the conserved regions described herein. 1×SSC is 0.15M NaCl and 15 mM sodium citrate; the hybridisation solution and wash solutions may additionally include 5×Denhardt's reagent, 0.5-1.0% SDS, 100 μg/ml denatured, fragmented salmon sperm DNA, 0.5% sodium pyrophosphate. Another example of high stringency conditions is hybridisation at 65° C. in 0.1×SSC comprising 0.1 SDS and optionally 5×Denhardt's reagent, 100 μg/ml denatured, fragmented salmon sperm DNA, 0.5% sodium pyrophosphate, followed by the washing at 65° C. in 0.3×SSC.

For the purposes of defining the level of stringency, reference can be made to Sambrook et al. (2001) Molecular Cloning: a laboratory manual, 3rd Edition, Cold Spring Harbor Laboratory Press, CSH, New York or to Current Protocols in Molecular Biology, John Wiley & Sons, N.Y. (1989 and yearly updates).

The term “nucleic acid construct” as used herein refers to a nucleic acid molecule, either single- or double-stranded, which is isolated from a naturally occurring gene or is modified to contain segments of nucleic acids in a manner that would not otherwise exist in nature or is synthetic.

The term “nucleic acid construct” is synonymous with the term “expression cassette” when the nucleic acid construct contains the control sequences required for expression of a polynucleotide.

The term “control sequence” or “genetic control element” is defined herein to include all sequences affecting the expression of a polynucleotide, including but not limited thereto, the expression of a polynucleotide encoding a polypeptide. Each control sequence may be native or foreign to the polynucleotide or native or foreign to each other. Such control sequences include, but are not limited to, promoter sequence, 5′-UTR (also called leader sequence), ribosomal binding site (RBS), 3′-UTR, and transcription start and stop sites.

The term “functional linkage” or “operably linked” with respect to regulatory elements, is to be understood as meaning the sequential arrangement of a regulatory element (including but not limited thereto a promoter) with a nucleic acid sequence to be expressed and, if appropriate, further regulatory elements (including but not limited thereto a terminator) in such a way that each of the regulatory elements can fulfil its intended function to allow, modify, facilitate or otherwise influence expression of said nucleic acid sequence. For example, a control sequence is placed at an appropriate position relative to the coding sequence of the polynucleotide sequence such that the control sequence directs the expression of the coding sequence of a polypeptide.

A “promoter” or “promoter sequence” is a nucleotide sequence located upstream of a gene on the same strand as the gene that enables that gene's transcription. A promoter is generally followed by the transcription start site of the gene. A promoter is recognized by RNA polymerase (together with any required transcription factors), which initiates transcription. A functional fragment or functional variant of a promoter is a nucleotide sequence which is recognizable by RNA polymerase, and capable of initiating transcription.

As used herein, the term “isolated DNA molecule” refers to a DNA molecule at least partially separated from other molecules normally associated with it in its native or natural state. The term “isolated” preferably refers to a DNA molecule that is at least partially separated from some of the nucleic acids which normally flank the DNA molecule in its native or natural state. Thus, DNA molecules fused to regulatory or coding sequences with which they are not normally associated, for example as the result of recombinant techniques, are considered isolated herein. Such molecules are considered isolated when integrated into the chromosome of a host cell or present in a nucleic acid solution with other DNA molecules, in that they are not in their native state.

Any number of methods well known to those skilled in the art can be used to isolate and manipulate a polynucleotide, or fragment thereof, as disclosed herein. For example, polymerase chain reaction (PCR) technology can be used to amplify a particular starting polynucleotide molecule and/or to produce variants of the original molecule. Polynucleotide molecules, or fragment thereof, can also be obtained by other techniques, such as by directly synthesizing the fragment by chemical means, as is commonly practiced by using an automated oligonucleotide synthesizer. A polynucleotide can be single-stranded (ss) or double-stranded (ds). “Double-stranded” refers to the base-pairing that occurs between sufficiently complementary, anti-parallel nucleic acid strands to form a double-stranded nucleic acid structure, generally under physiologically relevant conditions. Embodiments of the method include those wherein the polynucleotide is at least one selected from the group consisting of sense single-stranded DNA (ssDNA), sense single-stranded RNA (ssRNA), double-stranded RNA (dsRNA), double-stranded DNA (dsDNA), a double-stranded DNA/RNA hybrid, anti-sense ssDNA, or anti-sense ssRNA; a mixture of polynucleotides of any of these types can be used.

As used herein, “recombinant” when referring to nucleic acid or polypeptide, indicates that such material has been altered as a result of human application of a recombinant technique, such as by polynucleotide restriction and ligation, by polynucleotide overlap-extension, or by genomic insertion or transformation. A gene sequence open reading frame is recombinant if (a) that nucleotide sequence is present in a context other than its natural one, for example by virtue of being (i) cloned into any type of artificial nucleic acid vector or (ii) moved or copied to another location of the original genome, or if (b) the nucleotide sequence is mutagenized such that it differs from the wild type sequence. The term recombinant also can refer to an organism having a recombinant material, e.g., a plant that comprises a recombinant nucleic acid is a recombinant plant.

The term “transgenic” refers to an organism, preferably a plant or part thereof, or a nucleic acid that comprises a heterologous polynucleotide. Preferably, the heterologous polynucleotide is stably integrated within the genome such that the polynucleotide is passed on to successive generations. The heterologous polynucleotide may be integrated into the genome alone or as part of a recombinant expression cassette. “Transgenic” is used herein to refer to any cell, cell line, callus, tissue, plant part or plant, the genotype of which has been so altered by the presence of heterologous nucleic acid including those transgenic organisms or cells initially so altered, as well as those created by crosses or asexual propagation from the initial transgenic organism or cell. A “recombinant” organism preferably is a “transgenic” organism. The term “transgenic” as used herein is not intended to encompass the alteration of the genome (chromosomal or extra-chromosomal) by conventional plant breeding methods (e.g., crosses) or by naturally occurring events such as, e.g., self-fertilization, random cross-fertilization, non-recombinant viral infection, non-recombinant bacterial transformation, non-recombinant transposition, or spontaneous mutation.

As used herein, “mutagenized” refers to an organism or nucleic acid thereof having alteration(s) in the biomolecular sequence of its native genetic material as compared to the sequence of the genetic material of a corresponding wildtype organism or nucleic acid, wherein the alteration(s) in genetic material were induced and/or selected by human action. Examples of human action that can be used to produce a mutagenized organism or DNA include, but are not limited to treatment with a chemical mutagen such as EMS and subsequent selection with herbicide(s); or by treatment of plant cells with x-rays and subsequent selection with herbicide(s). Any method known in the art can be used to induce mutations. Methods of inducing mutations can induce mutations in random positions in the genetic material or can induce mutations in specific locations in the genetic material (i.e., can be directed mutagenesis techniques), such as by use of a genoplasty technique. In addition to unspecific mutations, according to the invention a nucleic acid can also be mutagenized by using mutagenesis means with a preference or even specificity for a particular site, thereby creating an artificially induced heritable allele according to the present invention. Such means, for example site specific nucleases, including for example zinc finger nucleases (ZFNs), meganucleases, transcription activator-like effector nucleases (TALENS) (Malzahn et al., Cell Biosci, 2017, 7:21) and clustered regularly interspaced short palindromic repeats/CRISPR-associated nuclease (CRISPR/Cas) with an engineered crRNA/tracr RNA (for example as a single-guide RNA, or as modified crRNA and tracrRNA molecules which form a dual molecule guide), and methods of using this nucleases to target known genomic locations, are well known in the art (see reviews by Bortesi and Fischer, 2015, Biotechnology Advances 33: 41-52; and by Chen and Gao, 2014, Plant Cell Rep 33: 575-583, and references within).

As used herein, a “genetically modified organism” (GMO) is an organism whose genetic characteristics contain alteration(s) that were produced by human effort causing transfection that results in transformation of a target organism with genetic material from another or “source” organism, or with synthetic or modified-native genetic material, or an organism that is a descendant thereof that retains the inserted genetic material. The source organism can be of a different type of organism (e.g., a GMO plant can contain bacterial genetic material) or from the same type of organism (e.g., a GMO plant can contain genetic material from another plant).

As used herein, “wildtype” or “corresponding wildtype plant” means the typical form of an organism or its genetic material, as it normally occurs, as distinguished from e.g. mutagenized and/or recombinant forms. Similarly, by “control cell”, “wildtype” “control plant, plant tissue, plant cell or host cell” is intended a plant, plant tissue, plant cell, or host cell, respectively, that lacks the particular polynucleotide of the invention that are disclosed herein. The use of the term “wildtype” is not, therefore, intended to imply that a plant, plant tissue, plant cell, or other host cell lacks recombinant DNA in its genome, and/or does not possess fungal resistance characteristics that are different from those disclosed herein.

As used herein, “descendant” refers to any generation plant. A progeny or descendant plant can be from any filial generation, e.g., F1, F2, F3, F4, F5, F6, F7, etc. In some embodiments, a descendant or progeny plant is a first, second, third, fourth, fifth, sixth, seventh, eighth, ninth, or tenth generation plant.

The term “plant” is used herein in its broadest sense as it pertains to organic material and is intended to encompass eukaryotic organisms that are members of the taxonomic kingdom plantae, examples of which include but are not limited to monocotyledon and dicotyledon plants, vascular plants, vegetables, grains, flowers, trees, herbs, bushes, grasses, vines, ferns, mosses, fungi and algae, etc, as well as clones, offsets, and parts of plants used for asexual propagation (e.g. cuttings, pipings, shoots, rhizomes, underground stems, clumps, crowns, bulbs, corms, tubers, rhizomes, plants/tissues produced in tissue culture, etc.). Unless stated otherwise, the term “plant” refers to a whole plant, any part thereof, or a cell or tissue culture derived from a plant, comprising any of: whole plants, plant components or organs (e.g., leaves, stems, roots, etc.), plant tissues, seeds, plant cells, and/or progeny of the same. A plant cell is a biological cell of a plant, taken from a plant or derived through culture from a cell taken from a plant.

Plants that are particularly useful in the methods of the invention include all plants which belong to the superfamily Viridiplantae, in particular monocotyledonous and dicotyledonous plants including fodder or forage legumes, ornamental plants, food crops, trees or shrubs selected from the list comprising Acer spp., Actinidia spp., Abelmoschus spp., Agave sisalana, Agropyron spp., Agrostis stolonifera, Allium spp., Amaranthus spp., Ammophila arenaria, Ananas comosus, Annona spp., Apium graveolens, Arachis spp., Artocarpus spp., Asparagus officinalis, Avena spp. (e.g. Avena sativa, Avena fatua, Avena byzantina, Avena fatua var. sativa, Avena hybrida), Averrhoa carambola, Bambusa sp., Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Cannabis sativa, Capsicum spp., Carex elata, Carica papaya, Carissa macrocarpa, Carya spp., Carthamus tinctorius, Castanea spp., Ceiba pentandra, Cichorium endivia, Cinnamomum spp., Citrullus lanatus, Citrus spp., Cocos spp., Coffea spp., Colocasia esculenta, Cola spp., Corchorus sp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eragrostis tef, Erianthus sp., Eriobotrya japonica, Eucalyptus sp., Eugenia uniflora, Fagopyrum spp., Fagus spp., Festuca arundinacea, Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus sinensis, Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Panicum virgatum, Passiflora edulis, Pastinaca sativa, Pennisetum sp., Persea spp., Petroselinum crispum, Phalaris arundinacea, Phaseolus spp., Phleum pratense, Phoenix spp., Phragmites australis, Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Tripsacum dactyloides, Triticosecale rimpaui, Triticum spp. (e.g. Triticum aestivum, Triticum durum, Triticum turgidum, Triticum hybernum, Triticum macha, Triticum sativum, Triticum monococcum or Triticum vulgare), Tropaeolum minus, Tropaeolum majus, Vaccinium spp., Vicia spp., Vigna spp., Viola odorata, Vitis spp., Zea mays, Zizania palustris, Ziziphus spp., amaranth, artichoke, asparagus, broccoli, Brussels sprouts, cabbage, canola, carrot, cauliflower, celery, collard greens, flax, kale, lentil, oilseed rape, okra, onion, potato, rice, soybean, strawberry, sugar beet, sugar cane, sunflower, tomato, squash, tea and algae, amongst others. According to a preferred embodiment of the present invention, the plant is a crop plant. Examples of crop plants include inter alia soybean, sunflower, canola, alfalfa, rapeseed, cotton, tomato, potato or tobacco.

According to the invention, a plant is cultivated to yield plant material. Cultivation conditions are chosen in view of the plant and may include, for example, any of growth in a greenhouse, growth on a field, growth in hydroculture and hydroponic growth.

The plant, hereinafter also called “yield improvement plant”, comprises a gene selected from Pti5, SAR8.2 and RLK2. It has now surprisingly been found that these genes can convey improved yield both under standard growth conditions established in the respective region of plantation and under pathogen challenged growth conditions, in particular under fungal pathogen prevalence in the general area where the plants are grown.

Plants comprising a Pti5, SAR8.2 or RLK2 gene have been described before, among others, in WO2013001435, WO2014076614 and WO2014024102. The documents, however, does not show any improvement of yield. Instead, they focus on the achievement of fungal resistance. As shown herein, however, fungal resistance is no predictor for yield improvements. Thus, these documents only provide a general technical background concerning certain plants comprising the aforementioned genes but do not imply or even render likely that any yield improvement as described by the present invention can be achieved.

For the purposes of the invention, a Pti5 gene codes for a protein comprising, among others, an apetala 2 domain as explained in PFAM entry PF00847 and binding to the Pti5 GCC box as described by Gu et al 2002 The Plant Cell, Vol. 14, 817-831. Preferably, the Pti5 gene codes for a protein whose amino acid sequence has at least 40%, more preferably at least 43%, more preferably at least 50%, more preferably at least 58%, more preferably at least 67%, more preferably at least 70%, more preferably at least 71% sequence identity to SEQ ID NO. 1, wherein preferably the sequence identity to SEQ ID NO. 1 is at most 80%, more preferably at most 79%. Particularly preferred are thus plants expressing a Pti5 gene whose corresponding polypeptide sequence has 58-80% sequence identity to SEQ ID NO. 1, more preferably 67-79% sequence identity to SEQ ID NO. 1. It is to be understood that SEQ ID NO. 1 is an artificial amino acid sequence specifically constructed as a template for amino acid sequence annealing purposes. The sequence can thus be used for identification of Pti5 genes independent from the fact that no Pti5 activity of the polypeptide of SEQ ID NO. 1 is shown herein. Particularly preferred as a Pti5 gene in a method or plant according to the present invention is any of the amino acid sequences defined by the following Uniprot identifiers: PTI5_SOLLC, M1AQ94_SOLTU, A0A2G3A6U8_CAPAN, A0A2G2XEI7_CAPBA, A0A2G3D5K5_CAPCH, A0A1S4BF73_TOBAC, A0A1U7WC00_NICSY, A0A1S4A5G9_TOBAC, A0A1J6J1M1_NICAT, A0A1S2X9U7_CICAR, G7IFJ0_MEDTR, A0A2K3KXT4_TRIPR, V7BQ20_PHAVU, A0A1S3VIX3_VIGRR, A0A0L9VF85_PHAAN, A0A445GQU3_GLYSO, A0A0R0G4Q5_SOYBN, A0A061GM02_THECC, A0A44518U7_GLYSO, A0A0D2S2G5_GOSRA, A0A4P1QVV4_LUPAN, A0A151SAR8.21_CAJCA, A0A2J6MBZ7_LACSA, A0A2K3LDZ4_TRIPR, A0A2U1QDE9_ARTAN, A0A444WYK6_ARAHY. Particularly preferred according to the invention are Pti5 genes, and plants expressing them, which code for a polypeptide having at least 60%, more preferably at least 71%, more preferably at least 75%, more preferably at least 79%, more preferably at least 82%, more preferably at least 90% sequence identity to the amino acid sequence given by Uniprot identifier PTI5_SOLLC.

For the purposes of the invention, a SAR8.2 gene codes for a protein comprising or consisting of a SAR8.2 domain as explained in PFAM entry PF03058. Preferably, the SAR8.2 gene codes for a protein whose amino acid sequence has at least 35%, more preferably at least 45%, more preferably at least 55%, more preferably at least 72%, more preferably at least 77%, more preferably at least 82%, more preferably at least 84%, more preferably at least 86%, more preferably at least 88%, more preferably at least 89% sequence identity to SEQ ID NO. 2, wherein preferably the sequence identity to SEQ ID NO. 2 is at most 98%, more preferably at most 95%. Particularly preferred are thus plants expressing a SAR8.2 gene whose corresponding polypeptide sequence has 72-98% sequence identity to SEQ ID NO. 1, more preferably 74-92% sequence identity to SEQ ID NO. 2. It is to be understood that SEQ ID NO. 2 is an artificial amino acid sequence specifically constructed as a template for amino acid sequence annealing purposes. The sequence can thus be used for identification of SAR8.2 genes independent from the fact that no SAR8.2 activity of the polypeptide of SEQ ID NO. 2 is shown herein. Particularly preferred as a SAR8.2 gene in a method or plant according to the present invention is any of the amino acid sequences defined by the following Uniprot identifiers: Q8W2C1_CAPAN, Q9SEM2_CAPAN, A0A2G2X990_CAPBA, Q947G6_CAPAN, Q947G5_CAPAN, A0A2G2X9U8_CAPBA, A0A2G3CEJ1_CAPCH, A0A2G2X931_CAPBA, M1BEK3_SOLTU, A0A3Q7J4M2_SOLLC, A0A2G2ZTB6_CAPAN, A0A2G3CRF6_CAPCH, A0A2G2W296_CAPBA, A0A2G2WZ87_CAPBA, M1BIQ9_SOLTU, M1D489_SOLTU, M1D488_SOLTU, A0A2G2ZQ02_CAPAN, A0A1S4AM24_TOBAC, A0A1U7XJ42_NICSY, A0A1S4CJX7_TOBAC. Particularly preferred according to the invention are SAR8.2 genes, and plants expressing them, which code for a polypeptide having at least 60%, more preferably at least 68%, more preferably at least 88%, more preferably at least 91%, more preferably at least 95% sequence identity to the amino acid sequence given by Uniprot identifier Q8W2C1_CAPAN.

For the purposes of the invention, an RLK2 gene codes for a protein comprising a protein tyrosine kinase domain as explained in PFAM entry PF07714. Preferably, the RLK2 gene codes for a protein whose amino acid sequence has at least 60%, more preferably at least 65%, more preferably at least 69%, more preferably at least 72%, more preferably at least 77%, more preferably at least 81% sequence identity to SEQ ID NO. 3, wherein preferably the sequence identity to SEQ ID NO. 3 is at most 90%, more preferably at most 85%. Particularly preferred are thus plants expressing an RLK2 gene whose corresponding polypeptide sequence has 66-90% sequence identity to SEQ ID NO. 1, more preferably 72-85% sequence identity to SEQ ID NO. 3. It is to be understood that SEQ ID NO. 3 is an artificial amino acid sequence specifically constructed as a template for amino acid sequence annealing purposes. The sequence can thus be used for identification of RLK2 genes independent from the fact that no RLK2 activity of the polypeptide of SEQ ID NO. 3 is shown herein. Particularly preferred as an RLK2 gene in a method or plant according to the present invention is any of the amino acid sequences defined by the following Uniprot identifiers: Q9FLL2_ARATH, D7MIX9_ARALL, R0H5G6_9BRAS, V4LSN6_EUTSA, A0A0D3CT78_BRAOL, A0A397YSZ3_BRACM, A0A078JM18_BRANA, M4E174_BRARP, A0A2J6M2D4_LACSA, A0A2U1NZW7_ARTAN, A0A251SV29_HELAN, A0A251T618_HELAN, A0A444ZYR1_ARAHY, 11K6K6_SOYBN, A0A445KRF2_GLYSO, A0A0S3T624_PHAAN, V7CJW2_PHAVU, A0A1S3VSF7_VIGRR, A0A061G564_THECC, A0A1R3IAA5_COCAP, A0A1R3GKT2_9ROSI, A0A0D2SO45_GOSRA, A0A1U8LSG2_GOSHI, A0A1S3Z3A6_TOBAC, A0A1J6KIE1_NICAT, A0A1S4API7_TOBAC, A0A1U7VRW3_NICSY, A0A3Q7HTK8_SOLLC, A0A2G2XL26_CAPBA, A0A2G3AE00_CAPAN, M1AWD0_SOLTU, A0A2G3B3F6_CAPCH, M1A1Q9_SOLTU.

Particularly preferred according to the invention are RLK2 genes, and plants expressing them, which code for a polypeptide having at least 55%, more preferably at least 72%, more preferably at least 80, more preferably at least 87%, more preferably at least 92% sequence identity to the amino acid sequence given by Uniprot identifier Q9FLL2_ARATH.

According to the present invention, the plant comprises an expression cassette, wherein the expression cassette comprises said gene selected from Pti5, SAR8.2 and RLK2. According to the invention, an expression cassette comprises the respective gene and the control sequences required for expression of the gene. Preferably an expression cassette comprises at least a promoter and, operably linked thereto, the gene selected from Pti5, SAR8.2 and RLK2. More preferably, the expression cassette also comprises a terminator in 3′ direction downstream of the respective gene. Exemplary expression cassettes are disclosed, for example, in the aforementioned documents WO2013001435, WO2014076614 and WO2014024102, in particular those comprising the sequences SEQ ID NO. 6, 3 and 10, respectively. Those expression cassettes and corresponding description are incorporated herein by reference.

The expression cassette is a heterologous expression cassette. According to the invention, the expression cassette is “heterologous” if any of the following conditions is fulfilled: (1) The gene codes for a polypeptide (Pti5, SAR8.2, RLK2, respectively) with a sequence different to the wild type plant; (2) the gene is under control of a promoter not present in the wild type plant or not connected to the gene in the wild type plant; (3) the expression cassette is integrated at a different locus in the plant genome compared to the wild type plant. Thus, the yield improvement plants used according to the present invention preferably are transgenic plants. Furthermore, the methods according to the present invention preferably exclude plants exclusively obtained by means of an essentially biological process, e.g. the crossing of gametes found in nature. This preferred exclusion has no technical reason but is exclusively intended to appease the legislator and vocal NGOs. Preferably not excluded, however, are plants obtained by crossing and selection of at least one transgenic plant and another plant, as long as the offspring comprises the heterologous expression cassette.

The plants are grown under appropriate conditions. Growth of the plants according to the present invention leads to improved yield, wherein growth preferably is under low pathogen pressure. For describing the present invention the term “low pathogen pressure” denotes the normal pathogen pressure of an average growth season at the respective location, more preferably the average pathogen pressure at an average growth season in Mato Grosso. When pathogen pressure is higher than such low pathogen pressure, it is preferred to treat the plants with a fungicide to keep the number of visible lesions below half of what is observed in a non-fungicide treated control plant. However, it is an advantage of the present invention that yield can also be increased under higher pathogen pressure, as will be shown in the examples below. It is a particular advantage of the present invention that the plants can be cultivated using any of the established applicable cultivation techniques established in the art. Thus, the invention advantageously provides a method applicable under the broadest variety of cultivation conditions including growth on a field and in a greenhouse. Thus, the use of each of the genes Pti5, SAR8.2 and RLK2 to improve yield under all pathogen pressure conditions is surprisingly versatile.

According to the invention the yield is preferably one or more of
biomass per area,
grain mass per area,
seed mass per area.

As used herein, “yield” refers to the amount of agricultural production harvested per unit of land. Yield can be any of total harvested biomass per area, total harvested grain mass per area and total harvested seed mass per area. Yield is measured by any unit, for example metric ton per hectare or bushels per acre. Yield is adjusted for moisture of harvested material, wherein moisture is measured at harvest in the harvested biomass, grain or seed, respectively. For example, moisture of soybean seed is preferably 15%.

As described above, yield improvement is measured in comparison to the yield obtained by a control plant. The control plant is a plant lacking the expression cassette referred to above, but is otherwise cultivated under identical conditions. Improvement of yield is determined by the yield of a “yield improvement plant” comprising said heterologous expression cassettes relative to a control plant of the same species or, if applicable, variety, wherein the control plant does not comprise said heterologous expression cassette.

It is to be understood that when reference is made to yield or treatment of “a plant”, it is preferred not to determine the yield or perform the treatment on a single plant compared to a single control plant. Instead, yield is determined by the yield obtained from an ensemble of the plants, preferably an ensemble of at least 1000 plants, preferably wherein the plants are cultivated on a field or in a greenhouse. Most preferably the yield of a monoculture field of at least 1 ha of the plant and a monoculture field of at least 1 ha of the control plant is determined, respectively. Correspondingly, treatments are preferably performed on such ensemble of plants.

In view of the above advantages the invention also provides a farming method for improving the yield produced by a plant relative to a control plant, comprising cultivation of a plant comprising a heterologous expression cassette comprising a gene selected from Pti5, SAR8.2 and RLK2, wherein during cultivation of the plant the number of pesticide treatments per growth season is reduced by at least one relative to the control plant, preferably by at least two. Pesticide treatment schemes are generally established in standard agricultural practice for each region of plant growth. For example, in Brazil it may be customary to apply a first fungicide treatment to soybean plants on day 8 after seeding and a second spray on day 16 after seeding. In other regions a scheme may be practiced not depending on mere time of growth but, for example, taking into account first notice of a pest occurrence or passing of a pest incidence threshold. It is a particular and unforseen advantage of the present invention that the number of pesticide treatments per growth seasons can be reduced compared to a control plant. It was in particular surprising that such treatment reduction is possible not only without reducing yield; instead the farming method according to the invention advantageously allows to maintain or even increase yield despite the reduction in treatments. This greatly improves cost efficiency of farming the plants as provided by the present invention. Of course the pesticide is preferably applied in pesticidally effective amounts.

According to the invention the methods provided herein preferably provide an increased yield, relative to a control plant, in the absence or, more preferably, in the presence of a pathogen (also called “pest” herein). It is a particular advantage that the yield increase according to the invention not only can be achieved in a variety of climate conditions conductive for plant cultivation; the yield increase according to the invention has also consistently been found under most conditions. According to the invention the trait “yield improvement” is thus remarkably resilient under pest stress conditions. According to the invention, stress factors other than pest induced stress are preferably taken care of by established cultivation techniques. For example, nitrogen starvation stress is preferably removed by fertilization, and water limitation stress is preferably alleviated by irrigation.

According to the invention the pest preferably is or comprises at least a fungal pest, preferably a biotrophic or heminecrotrophic fungus, more preferably a rust fungus. If during cultivation the plant is also under threat of stress by other pathogens, e.g. nematodes and insects, such other pests are preferably taken care of by respective pesticide treatments. Thus, according to the invention preferably the number of fungicide treatments is reduced as described above, irrespective of other pesticide treatments. The fungicide is preferably applied in fungicidally effective amounts. The fungicide can be mixed with other pesticides and ingredients preferably selected from insecticides, nematicides, and acaricides, herbicides, plant growth regulators, fertilizers. Preferred mixing partners are insecticides, nematicides and fungicides. It is particularly preferred to reduce, during cultivation of the plant, the number of fungicide treatments per growth season by at least one relative to the control plant, preferably by at least two. Fungicides may include 2-(thiocyanatomethylthio)-benzothiazole, 2-phenylphenol, 8-hydroxyquinoline sulfate, ametoctradin, amisulbrom, antimycin, Ampelomyces quisqualis, azaconazole, azoxystrobin, Bacillus subtilis, Bacillus subtilis strain QST713, benalaxyl, benomyl, benthiavalicarb-isopropyl, benzylaminobenzene-sulfonate (BABS) salt, bicarbonates, biphenyl, bismerthiazol, bitertanol, bixafen, blasticidin-S, borax, Bordeaux mixture, boscalid, bromuconazole, bupirimate, calcium polysulfide, captafol, captan, carbendazim, carboxin, carpropamid, carvone, chlazafenone, chloroneb, chlorothalonil, chlozolinate, Coniothyrium minitans, copper hydroxide, copper octanoate, copper oxychloride, copper sulfate, copper sulfate (tribasic), cuprous oxide, cyazofamid, cyflufenamid, cymoxanil, cyproconazole, cyprodinil, dazomet, debacarb, diammonium ethylenebis-(dithiocarbamate), dichlofluanid, dichlorophen, diclocymet, diclomezine, dichloran, diethofencarb, difenoconazole, difenzoquat ion, diflumetorim, dimethomorph, dimoxystrobin, diniconazole, diniconazole-M, dinobuton, dinocap, diphenylamine, dithianon, dodemorph, dodemorph acetate, dodine, dodine free base, edifenphos, enestrobin, enestroburin, epoxiconazole, ethaboxam, ethoxyquin, etridiazole, famoxadone, fenamidone, fenarimol, fenbuconazole, fenfuram, fenhexamid, fenoxanil, fenpiclonil, fenpropidin, fenpropimorph, fenpyrazamine, fentin, fentin acetate, fentin hydroxide, ferbam, ferimzone, fluazinam, fludioxonil, fluindapyr, flumorph, fluopicolide, fluopyram, fluoroimide, fluoxastrobin, fluquinconazole, flusilazole, flusulfamide, flutianil, flutolanil, flutriafol, fluxapyroxad, folpet, formaldehyde, fosetyl, fosetyl-aluminium, fuberidazole, furalaxyl, furametpyr, guazatine, guazatine acetates, GY-81, hexachlorobenzene, hexaconazole, hymexazol, imazalil, imazalil sulfate, imibenconazole, iminoctadine, iminoctadine triacetate, iminoctadine tris(albesilate), iodocarb, ipconazole, ipfenpyrazolone, iprobenfos, iprodione, iprovalicarb, isoprothiolane, isofetamide, isopyrazam, isotianil, kasugamycin, kasugamycin hydrochloride hydrate, kresoxim-methyl, laminarin, mancopper, mancozeb, mandipropamid, maneb, mefenoxam, mepanipyrim, mepronil, meptyl-dinocap, mercuric chloride, mercuric oxide, mercurous chloride, metalaxyl, metalaxyl-M, metam, metam-ammonium, metam-potassium, metam-sodium, metconazole, methasulfocarb, methyl iodide, methyl isothiocyanate, metiram, metominostrobin, metrafenone, mildiomycin, myclobutanil, nabam, nitrothal-isopropyl, nuarimol, octhilinone, ofurace, oleic acid (fatty acids), orysastrobin, oxadixyl, oxathiapiprolin, oxine-copper, oxpoconazole fumarate, oxycarboxin, pefurazoate, penconazole, pencycuron, penflufen, pentachlorophenol, pentachlorophenyl laurate, penthiopyrad, phenylmercury acetate, phosphonic acid, phthalide, picoxystrobin, polyoxin B, polyoxins, polyoxorim, potassium bicarbonate, potassium hydroxyquinoline sulfate, probenazole, prochloraz, procymidone, propamocarb, propamocarb hydrochloride, propiconazole, propineb, proquinazid, pydiflumetofen, prothioconazole, pyraclostrobin, pyrametostrobin, pyraoxystrobin, pyraziflumid, pyrazophos, pyribencarb, pyributicarb, pyrifenox, pyrimethanil, pyriofenone, pyroquilon, quinoclamine, quinoxyfen, quintozene, Reynoutria sachalinensis extract, sedaxane, silthiofam, simeconazole, sodium 2-phenylphenoxide, sodium bicarbonate, sodium pentachlorophenoxide, spiroxamine, sulfur, SYP-Z048, tar oils, tebuconazole, tebufloquin, tecnazene, tetraconazole, thiabendazole, thifluzamide, thiophanate-methyl, thiram, tiadinil, tolclofos-methyl, tolylfluanid, triadimefon, triadimenol, triazoxide, tricyclazole, tridemorph, trifloxystrobin, triflumizole, triforine, triticonazole, validamycin, valifenalate, valiphenal, vinclozolin, zineb, ziram, zoxamide, Candida oleophila, Fusarium oxysporum, Gliocladium spp., Phlebiopsis gigantea, Streptomyces griseoviridis, Trichoderma spp., (RS)-N-(3,5-dichlorophenyl)-2-(methoxymethyl)-succinimide, 1,2-dichloropropane, 1,3-dichloro-1,1,3,3-tetrafluoroacetone hydrate, 1-chloro-2,4-dinitronaphthalene, 1-chloro-2-nitropropane, 2-(2-heptadecyl-2-imidazolin-1-yl)ethanol, 2,3-dihydro-5-phenyl-1,4-dithi-ine 1,1,4,4-tetraoxide, 2-methoxyethylmercury acetate, 2-methoxyethylmercury chloride, 2-methoxyethylmercury silicate, 3-(4-chlorophenyl)-5-methylrhodanine, 4-(2-nitroprop-I-enyl)phenyl thiocyanateme, aminopyrifen, ampropylfos, anilazine, azithiram, barium polysulfide, Bayer 32394, benodanil, benquinox, bentaluron, benzamacril; benzamacril-isobutyl, benzamorf, benzovindiflupyr, binapacryl, bis(methylmercury) sulfate, bis(tributyltin) oxide, buthiobate, cadmium calcium copper zinc chromate sulfate, carbamorph, CECA, chlobenthiazone, chloraniformethan, chlorfenazole, chlorquinox, climbazole, copper bis(3-phenylsalicylate), copper zinc chromate, coumoxystrobin, cufraneb, cupric hydrazinium sulfate, cuprobam, cyclafuramid, cypendazole, cyprofuram, decafentin, dichlobentiazox, dichlone, dichlozoline, diclobutrazol, dimethirimol, dinocton, dinosulfon, dinoterbon, dipymetitrone, dipyrithione, ditalimfos, dodicin, drazoxolon, EBP, enoxastrobin, ESBP, etaconazole, etem, ethirim, fenaminosulf, fenaminstrobin, fenapanil, fenitropan, fenpicoxamid, fluindapyr, fluopimomide, fluotrimazole, flufenoxystrobin, furcarbanil, furconazole, furconazole-cis, furmecyclox, furophanate, glyodine, griseofulvin, halacrinate, Hercules 3944, hexylthiofos, ICIA0858, inpyrfluxam, ipfentrifluconazole, ipflufenoquin, isofetamid, isoflucypram, isopamphos, isovaledione, mandestrobin, mebenil, mecarbinzid, mefentrifluconazole, metazoxolon, methfuroxam, methylmercury dicyandiamide, metsulfovax, metyltetraprole, milneb, mucochloric anhydride, myclozolin, N-3,5-dichlorophenyl-succinimide, N-3-nitrophenylitaconimide, natamycin, N-ethylmercurio-4-toluenesulfonanilide, nickel bis(dimethyldithiocarbamate), OCH, oxathiapiprolin, phenylmercury dimethyldithiocarbamate, phenylmercury nitrate, phosdiphen, picarbutrazox, prothiocarb; prothiocarb hydrochloride, pydiflumetofen, pyracarbolid, pyrapropoyne, pyraziflumid, pyridachlometyl, pyridinitril, pyrisoxazole, pyroxychlor, pyroxyfur, quinacetol, quinacetol sulfate, quinazamid, quinconazole, quinofumelin, rabenzazole, salicylanilide, SSF-109, sultropen, tecoram, thiadifluor, thicyofen, thiochlorfenphim, thiophanate, thioquinox, tioxymid, triamiphos, triarimol, triazbutil, trichlamide, triclopyricarb, triflumezopyrim, urbacid, zarilamid, and any combinations thereof.

The pathogen according to the invention preferably is a fungus or a fungus-like organism from the phyla Ascomycota, Basisiomycota or Oomycota, more preferably of phylum Basidiomycota, even more preferably of subphylum Pucciniomycotina, even more preferably of class Pucciniomycetes, even more preferably of order Pucciniales, even more preferably of family Chaconiaceae, Coleosporiaceae, Cronartiaceae, Melampsoraceae, Mikronegeriaceae, Phakopsoraceae, Phragmidiaceae, Pileolariaceae, Pucciniaceae, Pucciniastraceae, Pucciniosiraceae, Raveneliaceae, Sphaerophragmiaceae or Uropyxidaceae,

even more preferably of genus Rhizoctonia, Maravalia, Ochropsora, Olivea, Chrysomyxa, Coleosporium, Diaphanopellis, Cronartium, Endocronartium, Peridermium, Melampsora, Chrysocelis, Mikronegeria, Arthuria, Batistopsora, Cerotelium, Dasturella, Phakopsora, Prospodium, Arthuriomyces, Catenulopsora, Gerwasia, Gymnoconia, Hamaspora, Kuehneola, Phragmidium, Trachyspora, Triphragmium, Atelocauda, Pileolaria, Racospermyces, Uromycladium, Allodus, Ceratocoma, Chrysocyclus, Cumminsiella, Cystopsora, Endophyllum, Gymnosporangium, Miyagia, Puccinia, Puccorchidium, Roestelia, Sphenorchidium, Stereostratum, Uromyces, Hyalopsora, Melampsorella, Melampsoridium, Milesia, Milesina, Naohidemyces, Pucciniastrum, Thekopsora, Uredinopsis, Chardoniella, Dietelia, Pucciniosira, Diorchidium, Endoraecium, Kernkampella, Ravenelia, Sphenospora, Austropuccinia, Nyssopsora, Sphaerophragmium, Dasyspora, Leucotelium, Macruropyxis, Porotenus, Tranzschelia or Uropyxis,
even more preferably of species Rhizoctonia alpina, Rhizoctonia bicornis, Rhizoctonia butinii, Rhizoctonia callae, Rhizoctonia carotae, Rhizoctonia endophytica, Rhizoctonia floccosa, Rhizoctonia fragariae, Rhizoctonia fraxini, Rhizoctonia fusispora, Rhizoctonia globularis, Rhizoctonia gossypii, Rhizoctonia muneratii, Rhizoctonia papayae, Rhizoctonia quercus, Rhizoctonia repens, Rhizoctonia rubi, Rhizoctonia silvestris, Rhizoctonia solani, Phakopsora ampelopsidis, Phakopsora apoda, Phakopsora argentinensis, Phakopsora cherimoliae, Phakopsora cingens, Phakopsora coca, Phakopsora crotonis, Phakopsora euvitis, Phakopsora gossypii, Phakopsora hornotina, Phakopsora jatrophicola, Phakopsora meibomiae, Phakopsora meliosmae, Phakopsora meliosmae-myrianthae, Phakopsora montana, Phakopsora muscadiniae, Phakopsora myrtacearum, Phakopsora nishidana, Phakopsora orientalis, Phakopsora pachyrhizi, Phakopsora phyllanthi, Phakopsora tecta, Phakopsora uva, Phakopsora vitis, Phakopsora ziziphi-vulgaris, Puccinia abrupta, Puccinia acetosae, Puccinia achnatheri-sibirici, Puccinia acroptili, Puccinia actaeae-agropyri, Puccinia actaeae-elymi, Puccinia antirrhini, Puccinia argentata, Puccinia arrhenatheri, Puccinia arrhenathericola, Puccinia artemisiae-keiskeanae, Puccinia arthrocnemi, Puccinia asteris, Puccinia atra, Puccinia aucta, Puccinia ballotiflora, Puccinia bartholomaei, Puccinia bistortae, Puccinia cacabata, Puccinia calcitrapae, Puccinia calthae, Puccinia calthicola, Puccinia calystegiae-soldanellae, Puccinia canaliculata, Puccinia caricis-montanae, Puccinia caricis-stipatae, Puccinia carthami, Puccinia cerinthes-agropyrina, Puccinia cesatii, Puccinia chrysanthemi, Puccinia circumdata, Puccinia clavata, Puccinia coleataeniae, Puccinia coronata, Puccinia coronati-agrostidis, Puccinia coronati-brevispora, Puccinia coronati-calamagrostidis, Puccinia coronati-hordei, Puccinia coronati-japonica, Puccinia coronati-longispora, Puccinia crotonopsidis, Puccinia cynodontis, Puccinia dactylidina, Puccinia dietelii, Puccinia digitata, Puccinia distincta, Puccinia duthiae, Puccinia emaculata, Puccinia erianthi, Puccinia eupatorii-columbiani, Puccinia flavenscentis, Puccinia gastrolobii, Puccinia geitonoplesii, Puccinia gigantea, Puccinia glechomatis, Puccinia helianthi, Puccinia heterogenea, Puccinia heterospora, Puccinia hydrocotyles, Puccinia hysterium, Puccinia impatientis, Puccinia impedita, Puccinia imposita, Puccinia infra-aequatorialis, Puccinia insolita, Puccinia justiciae, Puccinia klugkistiana, Puccinia knersvlaktensis, Puccinia lantanae, Puccinia lateritia, Puccinia latimamma, Puccinia liberta, Puccinia littoralis, Puccinia lobata, Puccinia lophatheri, Puccinia loranthicola, Puccinia menthae, Puccinia mesembryanthemi, Puccinia meyeri-albertii, Puccinia miscanthi, Puccinia miscanthidii, Puccinia mixta, Puccinia montanensis, Puccinia morata, Puccinia morthieri, Puccinia nitida, Puccinia oenanthes-stoloniferae, Puccinia operta, Puccinia otzeniani, Puccinia patriniae, Puccinia pentstemonis, Puccinia persistens, Puccinia phyllostachydis, Puccinia pittieriana, Puccinia platyspora, Puccinia pritzeliana, Puccinia prostii, Puccinia pseudodigitata, Puccinia pseudostriiformis, Puccinia psychotriae, Puccinia punctata, Puccinia punctiformis, Puccinia recondita, Puccinia rhei-undulati, Puccinia rupestris, Puccinia senecionis-acutiformis, Puccinia septentrionalis, Puccinia setariae, Puccinia silvatica, Puccinia stipina, Puccinia stobaeae, Puccinia striiformis, Puccinia striiformoides, Puccinia stylidii, Puccinia substriata, Puccinia suzutake, Puccinia taeniatheri, Puccinia tageticola, Puccinia tanaceti, Puccinia tatarinovii, Puccinia tetragoniae, Puccinia thaliae, Puccinia thlaspeos, Puccinia tillandsiae, Puccinia tiritea, Puccinia tokyensis, Puccinia trebouxi, Puccinia triticina, Puccinia tubulosa, Puccinia tulipae, Puccinia tumidipes, Puccinia turgida, Puccinia urticae-acutae, Puccinia urticae-acutiformis, Puccinia urticae-caricis, Puccinia urticae-hirtae, Puccinia urticae-inflatae, Puccinia urticata, Puccinia vaginatae, Puccinia virgata, Puccinia xanthii, Puccinia xanthosiae, Puccinia zoysiae,
more preferably of species Phakopsora pachyrhizi, Puccinia graminis, Puccinia striiformis, Puccinia hordei or Puccinia recondita, more preferably of genus Phakopsora and most preferably Phakopsora pachyrhizi. As indicated above, fungi of these taxa are responsible for grave losses of crop yield. This applies in particular to rust fungi of genus Phakopsora. It is thus an advantage of the present invention that the method allows to reduce fungicide treatments against Phrakopsora pachyrhizi as described herein.

It is preferred according to the invention that the plant is a crop plant, preferably a dikotyledon, more preferably a plant of order Fabales, more preferably a plant of family Fabaceae, more preferably a plant of Tribus Phaseoleae, more preferably of genus Amphicarpaea, Cajanus, Canavalia, Dioclea, Erythrina, Glycine, Arachis, Lathyrus, Lens, Pisum, Vicia, Vigna, Phaseolus or Psophocarpus, even more preferably of species Amphicarpaea bracteata, Cajanus cajan, Canavalia brasiliensis, Canavalia ensiformis, Canavalia gladiata, Dioclea grandiflora, Erythrina latissima, Phaseolus acutifolius, Phaseolus lunatus, Phaseolus maculatus, Psophocarpus tetragonolobus, Vigna angularis, Vigna mungo, Vigna unguiculata, Glycine albicans, Glycine aphyonota, Glycine arenaria, Glycine argyrea, Glycine canescens, Glycine clandestina, Glycine curvata, Glycine cyrtoloba, Glycine dolichocarpa, Glycine falcata, Glycine gracei, Glycine hirticaulis, Glycine lactovirens, Glycine latifolia, Glycine latrobeana, Glycine microphylla, Glycine peratosa, Glycine pindanica, Glycine pullenii, Glycine rubiginosa, Glycine stenophita, Glycine syndetika, Glycine tabacina, Glycine tomentella, Glycine gracilis, Glycine max, Glycine max×Glycine soja, Glycine soja, more preferably of species Glycine gracilis, Glycine max, Glycine max×Glycine soja, Glycine soja, most preferably of species Glycine max. As shown herein particularly good yield improvements are obtained for soybean.

The crop may comprise, in addition to the heterologous expression cassette, one or more further heterologous elements. For example, transgenic soybean events comprising herbicide tolerance genes are for example, but not excluding others, GTS 40-3-2, MON87705, MON87708, MON87712, MON87769, MON89788, A2704-12, A2704-21, A5547-127, A5547-35, DP356043, DAS44406-6, DAS68416-4, DAS-81419-2, GU262, SYHTOH2, W62, W98, FG72 and CV127; transgenic soybean events comprising genes for insecticidal proteins are for example, but not excluding others, MON87701, MON87751 and DAS-81419. Cultivated plants comprising a modified oil content have been created by using the transgenes: gm-fad2-1, Pj.D6D, Nc.Fad3, fad2-1A and fatb1-A. Examples of soybean events comprising at least one of these genes are: 260-05, MON87705 and MON87769.

Plants comprising such singular or stacked traits as well as the genes and events providing these traits are well known in the art. For example, detailed information as to the mutagenized or integrated genes and the respective events are available from websites of the organizations International Service for the Acquisition of Agrl. biotech Applications (ISAAA) (http://www.isaaa.org/gmapprovaldatabase) and the Center for Environmental Risk Assessment (CERA) (http://cera-qmc.org/GMCropDatabase). Further information on specific events and methods to detect them can be found for soybean events H7-1, MON89788, A2704-12, A5547-127, DP305423, DP356043, MON87701, MON87769, CV127, MON87705, DAS68416-4, MON87708, MON87712, SYHTOH2, DAS81419, DAS81419×DAS44406-6, MON87751 in WO04/074492, WO06/130436, WO06/108674, WO06/108675, WO08/054747, WO08/002872, WO09/064652, WO09/102873, WO10/080829, WO10/037016, WO11/066384, WO11/034704, WO12/051199, WO12/082548, WO13/016527, WO13/016516, WO14/201235.

The heterologous expression cassette according to the invention preferably comprises the gene selected from Pti5, SAR8.2 and RLK2 operably linked to any of
a) a constitutively active promoter,
b) a tissue-specific or tissue-preferred promoter,
c) a promoter inducible by exposition of the plant to a pest, preferably a fungal pest.

A constitutively active promoter allows to provide the plant with a basal expression of the Pti5, SAR8.2 or RLK2 gene, respectively. A promoter with tissue specificity or preference provides such basal expression only or predominantly in the respective tissue. And an inducible promoter allows for a fast upregulation of expression upon exposition of the plant to the pest, thereby providing a fast reaction. Most preferably the plant in the method according to the present invention comprises the gene selected from Pti5, SAR8.2 and RLK2 in two copies, wherein one copy is under control of a constitutively active promoter, a tissue-specific or tissue-preferred promoter, and the other copy is under control of an inducible promoter, preferably a promoter inducible by exposition to the fungal pathogen, most preferably Phakopsora pachyrhizi. This way a comparatively low basal expression of the gene is ascertained, conserving metabolic resources, while defenses against significant pest exposure are ramped up when needed, thereby consuming metabolic resources for gene expression mainly when there is a significant exposure to the stress.

The invention also provides a method for producing a hybrid plant having improved yield relative to a control plant, comprising
i) providing a first plant material comprising a heterologous expression cassette comprising a gene selected from Pti5, SAR8.2 and RLK2, and a second plant material not comprising said heterologous expression cassette,
ii) producing an F1 generation from a cross of the first and second plant material, and
iii) selecting one or more members of the F1 generation that comprises said heterologous expression cassette.

It is a particular advantage of the present invention that the methods of the present invention do not require homozygous plants expressing the gene selected from Pti5, SAR8.2 and RLK2 but is also applicable for hemizygous or heterozygous plants. Correspondingly the hybrid production method of the present invention advantageously provides hybrid plants comprising both the advantageous heterologous expression cassette of the present invention and advantageous traits of the second plant material. Thus the hybrid production method according to the present invention allows to construct, with low effort, hybrids adapted to expected growth conditions for the next growth season.

The invention is hereinafter further described by way of examples and selected preferred embodiments. Neither the examples nor the selected embodiments are intended to limit the scope of the claims.

### EXAMPLES

### Example 1: Obtaining of Transformed Soybean Plants

All steps leading to the generation and first evaluation of the transformed soybean plants described in this document, such as:
Isolation or synthesis of the respective genes
Generation of vectors for plant transformation
Transformation of the respective vectors in soybean plants
Evaluation of resistance of the transformed plants against soybean rust fungus are described in

WO2014118018 (resistance gene: EIN2), examples 2, 3 and 6

WO2013149804 (resistance gene: ACD), examples 2, 3 and 6

WO2013001435 (resistance gene: Pti5), examples 2, 3 and 6

WO2014076614 (resistance gene: SAR8.2), examples 2, 3 and 6

WO2014024079 (resistance gene: RLK2), examples 2, 3 and 6.

Where the above documents refer to more than one transformation method in example 3, the result in terms of yield and resistance to Phakopsora pachyrhizi were found independently of the transformation method employed.

Based on the result of the evaluation of resistance against soybean rust in TO and/or T1 generation, the most resistance and phenotypically best looking 3-5 events were selected for further analysis.

Homozygous T2 or T3 seeds were used for field trials. To obtain homozygous seeds, segregating T1 seeds I of the selected 3-5 events per construct were planted. Individual plants that were homozygous for the transgene were selected by using TaqMan® PCR assay as described by the manufacturer of the assay (Thermo Fisher Scientific, Waltham, MA USA 02451).

10-30 homozygous plants per event were grown under standard conditions (12 h daylength, 25° C.) and selfed (in-bred)Mature homozygous seeds were harvested approx. 120 days after planting. Harvested seeds of all 10-30 homozygous plants per event were pooled.

### Example 2: Field Trials

Homozygous seeds of 3-5 events per construct were tested in the field for resistance against soybean rust, yield and agronomic performance.

Field trials were performed in Brazil on up to three sites in the states of Sao Paulo, Minas Gerais and Mato Grosso. Field trials were planted depending on weather conditions in November or early December (Safra season) or early February (Safrinha season) to ensure inoculum of Asian soybean rust.

Material was tested in split plot trials (2 m long, 4 rows per plot), 3-4 replications per event and trial site. Field trials to test trait performance were grown using standard cultural practice, e.g. in terms of weed and insect control and fertilization but without any fungicide treatment to control fungal diseases.

About 10% of the plots were used as control. Depending on trial design the untransformed wild-type (WT) mother line or bulk of seeds harvested from null-segregants, grown in parallel to the transgenic mother plants (see above) were used as control.

### Example 3: ASR Rating

ASR infection was rated by experts using the scheme published by Godoy et al (2006) (citation Godoy, C., Koga, L., Canteri, M. (2006) Diagrammatic scale for assessment of soybean rust severity, Fitopatologia Brasileira 31(1)).

The three canopy levels (lower, middle and upper canopy) were rated independently and the average of the infection of all three canopy levels is counted as infection. At all 4-7 ratings were performed, starting at the early onset of disease and repeated mainly every 6-8 days. If weather was not suitable for disease progression the time in between 2 ratings was elongated.

To eliminate transgene insertion effects, which would be only dependent on the integration locus, 3 to 5 independent transgenic events were planted per field trial.

To compare the disease progression in different events over the season we calculated the Area Under Disease Progression Curve (AUDPC) (for reference see: M. J. Jeger and S. L. H. Viljanen-Rollinson (2001) The use of the area under the disease-progress curve (AUDPC) to assess quantitative disease resistance in crop cultivars Theor Appl Genet 102:32-40.)

To calculate the relative disease resistance the following formula was used

Relative disease resistance=(AUDPC(control)/AUDPC(event))−1)*100%

The relative disease resistance on gene (construct) level was calculated by averaging the relative disease resistance (based on formula above) of the 3-5 events expressing the same gene (=same construct).

As disease incidence and severity of soybean rust disease is strongly dependent on environmental conditions, field trials were performed at 2-3 different locations and up to 5 seasons. The significance of the result was calculated based on the average effect over the different locations and seasons.

The average relative disease resistance on gene/construct level in different locations and years is shown in the table below

| | Loc 1 | Loc 2 | Loc 1 | Loc 1 | Loc 2 | Loc 1 | Loc 2 | Loc 1 | Loc 2 | Loc 3 | |
|---|---|---|---|---|---|---|---|---|---|---|---|
| | Season 1 | Season 1 | Season 2 | Season 3 | Season 3 | Season 4 | Season 4 | Season 5 | Season 5 | Season 5 | Average |
| CaSAR | 2.9% | −3.6% | 13.9% | | | 0.7% | 5.1% | | | | 3.78% |
| Pti5 | | | | 86.0% | 13.7% | 31.6% | 5.7% | | | | 34.22% |
| RLK2 | | | −3.0% | 3.0% | −3.5% | 0.1% | −0.1% | −1.3% | 8.9% | −7.0% | −0.36% |
| EIN2 | | | | | | 11.8% | 16.9% | | | | 14.3% |
| ACD | | | | 5.0% | 2.9% | | | | | | 3.9% |

All genes, with the exception of RLK2 provided at least a slight increase of resistance under field conditions at the respective sites in the respective seasons.

### Example 4: Determination of Yield

For yield determination only the 2 middle rows per plot (see above) were harvested to reduce overestimation by edge effects. A combine was used which was able to record the total grain weight of the plot and grain moisture. After moisture correction the grain yield was calculated from kg/plot to kg/ha.

As most agronomic traits, such as yield, are strongly dependent on environmental conditions, field trials were performed at 2-3 different locations and up to 5 seasons. The significance of the result was calculated based on the average effect over the different locations and seasons.

| | Loc 1 | Loc 2 | Loc 1 | Loc 1 | Loc 2 | Loc 1 | Loc 2 | Loc 1 | Loc 2 | Loc 3 | |
|---|---|---|---|---|---|---|---|---|---|---|---|
| | Season 1 | Season 1 | Season 2 | Season 3 | Season 3 | Season 4 | Season 4 | Season 5 | Season 5 | Season 5 | Average |
| CaSAR | 9.3% | 50.5% | 26.5% | | | 41.1% | 16.0% | | | | 28.7% |
| Pti5 | | | | 4.3% | 4.5% | 14.0% | 10.5% | | | | 8.3% |
| RLK2 | | | 103.6% | 3.4% | 3.4% | 61.9% | 6.4% | 25.9% | 4.1% | 135.3% | 43.0% |
| EIN2 | | | | | | −2.9% | 22.8% | | | | −12.9% |
| ACD | | | | −10.5% | −24.7% | | | | | | −17.6% |

Overexpression of CaSAR, Pti5 and RLK2 significantly increased the yield generated by soybean when infected by soybean rust disease.

### Example 5: Analysis of Resistance and Yield Provided by the Different Genes

The ASR resistance provided by the expression of all different genes was analyzed in comparison with other agronomic traits, such as yield. To analyze the dependency (=correlation) between 2 factors a correlation analysis based on the formula developed by Pearson (Pearson's correlation; r) was performed. The formula is:

$$r = \frac{n ( \sum xy ) - ( \sum x ) ( \sum y )}{\sqrt{[ n \sum {x}^{2} - {( \sum x )}^{2} ] [ n \sum {y}^{2} - {( \sum y )}^{2} ]}}$$

With x are disease resistance values and y being the yield values from a specific season and location, and n the overall number of values (21).

The formula returns a value between −1 and 1, where: 1 indicates a strong positive relationship, −1 indicates a strong negative relationship and a result of zero indicates no relationship at all. All correlations below 0, 4 are generally considered as weak.

Using the formula above, the correlation factor of yield vs. resistance is −0,30, which indicates a very weak negative correlation of yield vs. resistance. This means higher resistance often results in lower yield, a fact that has also been described in literature before (see description section). Therefore it was surprising to also identify resistance genes that increase yield.

1. Method for improving the yield produced by a plant relative to a control plant, comprising
i) providing a plant comprising a heterologous expression cassette comprising a gene selected from Pti5, SAR8.2 and RLK2, and
ii) cultivating the plant.

2. Farming method for improving the yield produced by a plant relative to a control plant, comprising cultivation of a plant comprising a heterologous expression cassette comprising a gene selected from Pti5, SAR8.2 and RLK2, wherein during cultivation of the plant the number of pesticide treatments per growth season is reduced by at least one relative to the control plant.

3. Method according to claim 1, wherein
the yield is one or more of
biomass per area,
grain mass per area,
seed mass per area.

4. Method according to claim 1, wherein the yield is increased in the presence of a pest relative to a control plant.

5. Method according to claim 1, wherein the pest is or comprises at least a fungal pest.

6. Method according to claim 1, wherein the plant is a crop plant.

7. Method according to claim 1, wherein the heterologous expression cassette comprises the gene selected from Pti5, SAR8.2 and RLK2 operably linked to any of
a) a constitutively active promoter,
b) a tissue-specific or tissue-preferred promoter,
c) a promoter inducible by exposition of the plant to a pest.

8. Method according to claim 1, wherein the cultivation is performed on an ensemble of at least 1000 plants.

9. (canceled)

10. Method for producing a hybrid plant having improved yield relative to a control plant, comprising
i) providing a first plant material comprising a heterologous expression cassette comprising a gene selected from Pti5, SAR8.2 and RLK2, and a second plant material not comprising said heterologous expression cassette,
ii) producing an F1 generation from a cross of the first and second plant material, and
iii) selecting one or more members of the F1 generation that comprises said heterologous expression cassette.

11. The method of claim 2, wherein during cultivation of the plant the number of pesticide treatments per growth season is reduced by at least two relative to the control plant.

12. The method according to claim 2, wherein the pest is or comprises at least a biotrophic or heminecrotrophic fungus.

13. The method according to claim 2, wherein the pest is or comprises at least a rust fungus.

14. The method according to claim 6, wherein the plant is a dikotyledon.

15. The method according to claim 6, wherein the plant is a plant of order Fabales.

16. The method according to claim 6, wherein the plant is a plant of family Fabaceae.

17. The method according to claim 7, wherein the promoter is inducible by exposition of the plant to a fungal pest.

18. The method according to claim 8, wherein the plants are cultivated on a field or in a greenhouse.
